(19) United States
(12) Patent Application Publication
(10) Pub. No.: US 2025/0366917 A1
(43) Pub. Date: Dec. 4, 2025

(54) Laser-Treatment Device with Three Dimensional Mapping and Robotic Arm for Skin Lesions

(71) Applicant: Robert P. Thompson, Boerne, TX (US)

(72) Inventor: Robert P. Thompson, Boerne, TX (US)

(21) Appl. No.: 19/226,454

(22) Filed: Jun. 3, 2025

Related U.S. Application Data
(60) Provisional application No. 63/655,157, filed on Jun. 3, 2024.

(51) Int. Cl. A61B 18/20; A61B 18/00; A61B 34/00; A61B 34/10; A61B 34/32; G16H 10/60; G16H 20/40; G16H 40/63

(52) U.S. Cl. CPC A61B 18/203; A61B 34/10; A61B 34/25; A61B 34/32; A61B 34/70; G16H 10/60; G16H 20/40; G16H 40/63; A61B 2018/00178; A61B 2018/00184; A61B 2018/00452; A61B 2018/00702; A61B 2018/00732; A61B 2034/105; A61B 2034/107; A61B 2218/007

(57) ABSTRACT

A laser treatment device and method for dermatological and wound care using three-dimensional mapping and robotic arm guidance. A three-dimensional reconstruction of a targeted skin area is created using a scanning probe and a three-dimensional mapping application. Data from the three-dimensional reconstruction of the targeted skin area is compared with a database of skin treatment data to determine laser treatment instructions. A robotic arm delivers laser treatment to the targeted skin area according to the determined laser treatment instructions.

### CROSS-REFERENCES TO RELATED APPLICATIONS

This application claims the benefit of the filing date of provisional patent application No. 63/655,157 filed Jun. 3, 2024.

### STATEMENT REGARDING FEDERALLY SPONSORED RESEARCH OR DEVELOPMENT

Not applicable.

### BACKGROUND OF THE INVENTION

### 1. Field of the Invention

The present invention relates to a device and method for the treatment of skin lesions and other skin conditions. The treatment landscape for skin lesions, including cancer, wounds, and scar tissue, is evolving with advancements in laser therapies and three-dimensional mapping technologies. A skin lesion is any area of a person's skin that is abnormal from the skin around it. The present invention is an advancement in skin lesion treatment comprising three-dimensional reconstruction and precise laser treatment guided by a robotic arm, with all components combined into a singular comprehensive system. The devices and methods disclosed herein could also be used to provide other types of skin treatments including cosmetic skin care.

### BRIEF SUMMARY OF THE INVENTION

The present invention comprises an integrated three-dimensional mapping system, robotic arm, and laser. The components work together to provide precise laser therapy to a patient's skin by generating real-time three-dimensional data for the targeted skin lesion, communicating the real-time three-dimensional data, developing a laser treatment plan based on the three-dimensional data, and delivering tailored laser treatment to the targeted skin lesion based on the real-time three-dimensional data. The present invention can further include the use of artificial intelligence to increase the accuracy of the delivered treatments. The present invention can also be used to develop an artificial intelligence data set for laser skin treatment.

The present invention can be used to apply laser treatment to a variety of skin problems. Different skin problems require different colors and frequencies of laser strength. For example, laser tattoo removal is done with a different frequency and strength of laser than laser removal of skin cancer. There are also differences in the types of lasers used for cosmetic procedures such as skin resurfacing compared to more serious problems such as skin cancer and chronic ulcers. The invention described herein is not limited to any one specific type of laser treatment. The components and methods described herein can be used for a wide variety of medical laser treatments.

The present invention's combination of precision laser treatment with three-dimensional mapping and robotic arm guidance minimizes collateral damage to healthy tissue by the laser because the treatment is tailored based on the captured data set, which minimizes accidental and/or incidental treatment of non-targeted skin. This enhances treatment efficacy and patient comfort.

### DESCRIPTION OF THE SEVERAL VIEWS OF THE DRAWINGS

FIG. 1 is a perspective view of an embodiment of the laser treatment device.

FIG. 2 is an environmental view of an embodiment of the laser treatment device.

FIG. 3 is a diagram view of an embodiment of the computer interface of the laser treatment device.

FIG. 4 is a flow chart diagram of and embodiment of the method of performing laser treatment.

### DETAILED DESCRIPTION OF THE INVENTION

The present invention comprises a state-of-the art three-dimensional mapping and treatment system 20. Three-dimensional mapping is the generation of a three-dimensional profile of the surface of an object by processing an optical image of the object. Various devices, programs, and technology exist for providing three-dimensional mapping. A three-dimensional mapping application is a program that uses a scanning probe to create a three-dimensional reconstruction of a scanned target.

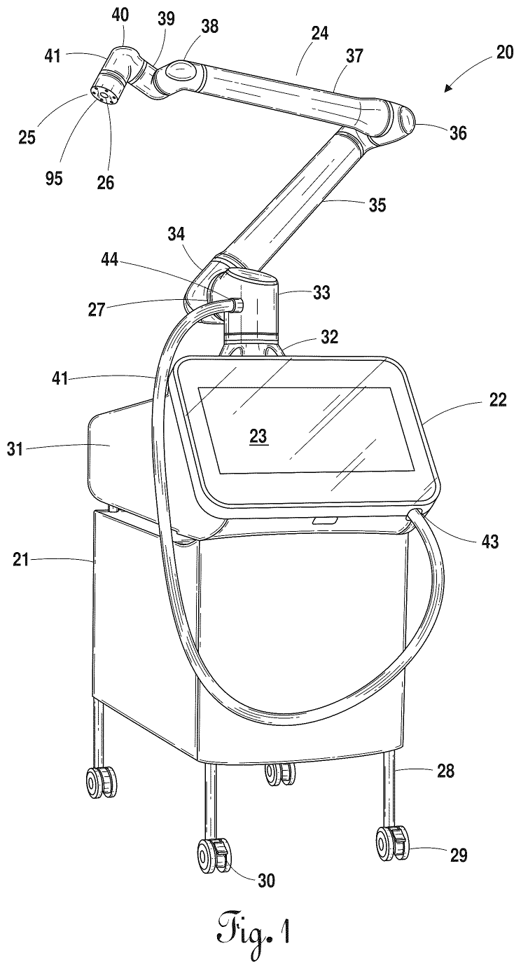

The structure of the three-dimensional mapping and treatment system 20 is described with reference to FIG. 1. The three-dimensional mapping and treatment system 20 comprises a base 21, a computer 22 with a computer interface 23, a robotic arm 24, a first laser 25, a vacuum 26, a second laser 27, legs 28, wheels 29, and a power source 48 (not shown).

In the embodiment described herein, the three-dimensional mapping and treatment system 20 comprises four legs 28. A wheel 29 is on the bottom end of each leg 28. Each wheel 29 comprises a locking mechanism 30. The base 21 sits on top of the legs 28. The base 21 can be used to store components such as circuitry, mechanical components, computer components, or spare equipment.

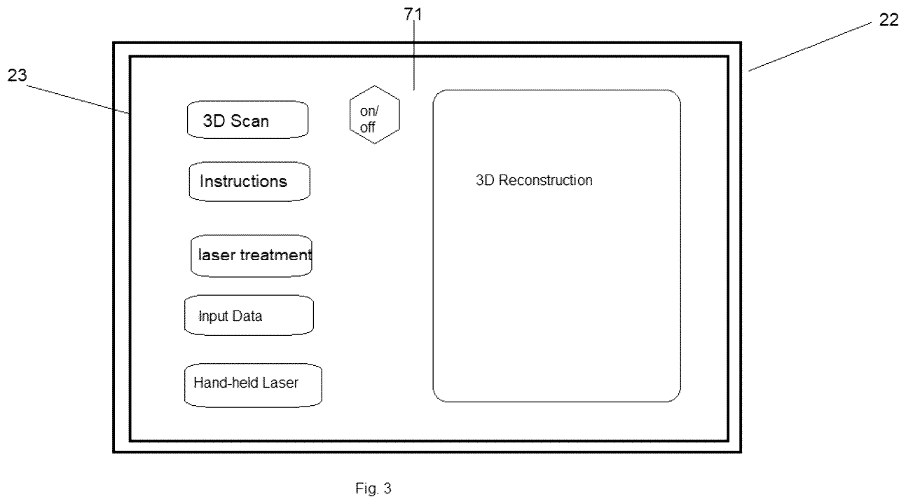

The computer 22 sits on top of the base 21. The computer 22 has a computer interface 23 that displays data in a format readable by a user. The computer interface 23 is optimally a touchscreen 71 wherein the user can operate and control the three-dimensional mapping and treatment system 20. Referring to FIG. 3, a mock-up of an embodiment of a computer interface 23 is shown. The computer is housed in a computer housing 31.

The robotic arm 24 protrudes from the top of the computer housing 31. The robotic arm 24 comprises a base portion 32, a first arm segment 33, a first elbow segment 34, a second arm segment 35, a second elbow segment 36, a third arm segment 37, a third elbow segment 38, a fourth arm segment 39, a fourth elbow segment 40, and a terminal end 41.

The terminal end 41 of the robotic arm 24 houses the first laser 25, a scanning probe 95, and a vacuum port 26. A HEPA filter 96 (not shown) is located in the interior of the three-dimensional mapping and treatment system 20. A conduit (not shown) connects the vacuum to the filter.

The second laser 27 is connected to the three-dimensional mapping and treatment system 20 via a cable 42. A first end 43 of the cable 42 is connected to the computer 22 via a port in the computer housing 31. A second end 44 of the cable 42 is connected to the second laser 27. The second laser 27 is removably secured to the first arm segment 33 of the robotic arm 24 via a docking port 45. For purposes of this application, removably secured means the second laser 27 can be detached and reattached to the robotic arm 24 without damaging the second laser 27 or the robotic arm 24 (or damaging any other component of the three-dimensional mapping and treatment system 20).

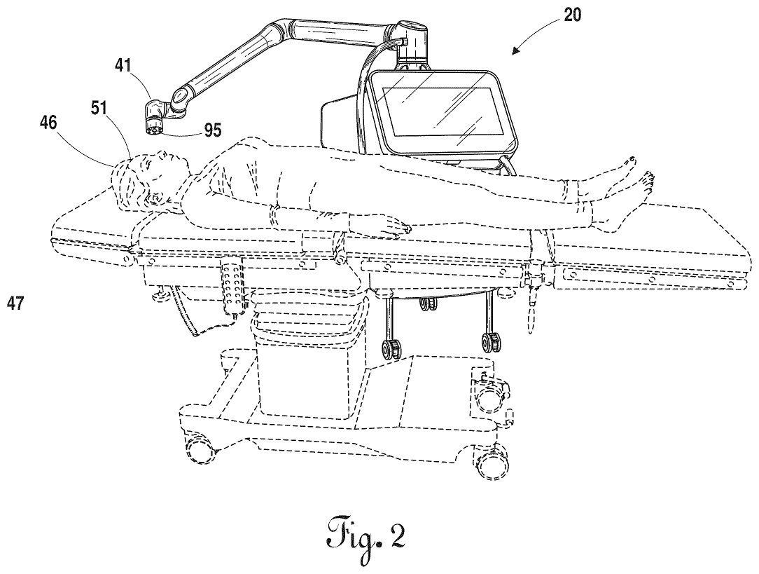

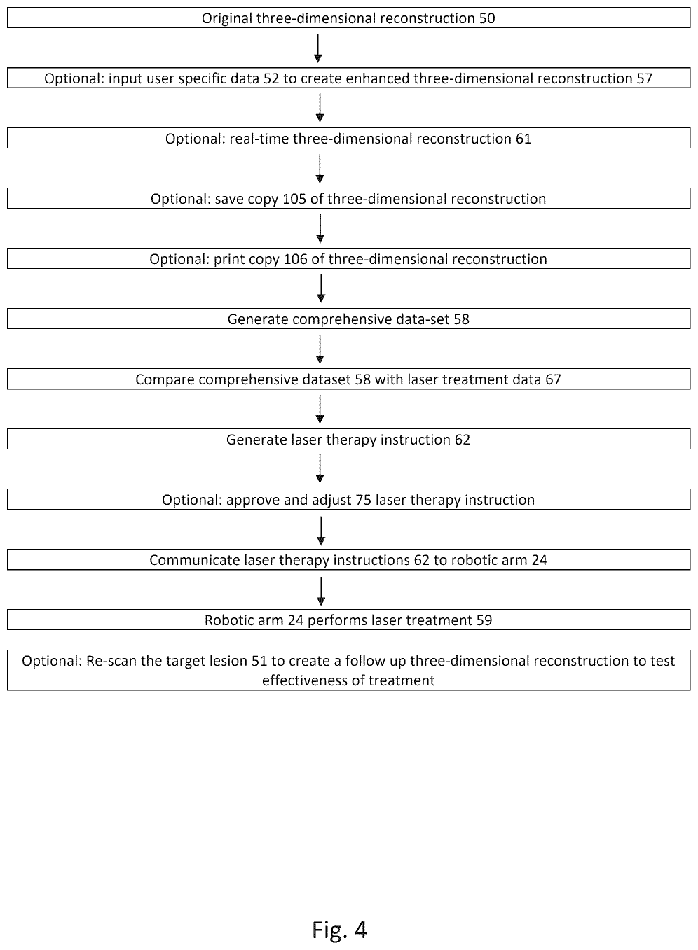

Operation of the three-dimensional mapping and treatment system 20 is described with reference to FIGS. 1-4. The three-dimensional mapping and treatment system 20 is maneuvered to a treatment site by rolling the unit 20 using the wheels 29. The treatment site could be an operating room, a doctor's office, a home, or any space suitable for providing laser treatment. Ideally, the treatment patient 46 is positioned on a platform 47 such as bed, table or stretcher. The three-dimensional mapping and treatment system 20 is connected to a power source 48. The three-dimensional mapping and treatment system 20 is turned on using an on/off switch. The on/off switch could be part of the touchscreen, or a physical on/off switch.

Persons or ordinary skill in the art understand that the maneuvering and power on steps described in the preceding paragraph do not need to be performed every time the unit is used. Sometimes, the unit will already be in the desired place and powered on.

The computer 22 of the three-dimensional mapping and treatment system 20 comprises a three-dimensional mapping application 49. The three-dimensional mapping application 49 could be software that is downloaded onto the computer 22, cloud-based software accessed wirelessly by the computer, coding that is stored on the hard drive of the computer 22, or a combination thereof. The three-dimensional mapping application 49 is integrated with the robotic arm 24. A scanning probe 95 is integral the terminal end 41 of the robotic arm 24.

The user manipulates the terminal end 41 of the robotic arm into close proximity with a targeted skin lesion 51 on the treatment patient 46. Normally, the scanning probe 95 will be positioned over the middle of the target lesion 51. In some embodiments, a circle may be drawn around the lesion to identify a targeted scan area. The user instructs the three-dimensional mapping and treatment system 20 to generate an original three-dimensional reconstruction 50 of the targeted skin lesion 51. The use may instruct the three-dimensional mapping and treatment system 20 to generate a three-dimensional reconstruction by selecting a three-dimensional scan option on the touch screen, or by any other suitable trigger mechanism.

When the user instructs the three-dimensional mapping and treatment system 20 to create a three-dimensional reconstruction, the scanning probe 95 scans the target skin lesion 51 and the three-dimensional mapping application 49 generates an original three-dimensional reconstruction 50 of the targeted skin lesion 51. The three-dimensional reconstruction 50 accounts for contours in the body. The three-dimensional reconstruction replicates the size and shape of the target lesion 51. The original three-dimensional reconstruction 50 of the targeted skin lesion is displayed on the computer interface 23. In some embodiments an image of the skin lesion could be captured with a separate scanning device and input into the three-dimensional mapping application 49. The three-dimensional mapping and treatment system 20 can store a copy of the original three-dimensional reconstruction 50. The three-dimensional mapping and treatment system comprises a save option 105 that allows the user to save a copy of the original three-dimensional reconstruction. The three-dimensional mapping and treatment system may also comprises a print 106 function that allows the user to print two-dimensional copies of the screen display.

The three-dimensional mapping and treatment system 20 can display a real-time three-dimensional reconstruction 61 of the targeted skin lesion 51 via the computer interface 23. The user 53 is able view the real-time three-dimensional reconstruction 61 while performing medical services. This allows the user 53 to observe changes to the skin lesion 51, via the real-time three-dimensional reconstruction 61, while performing treatment. The three-dimensional mapping and treatment system 20 allows the user to save a copy of the current version of the real-time three-dimensional reconstruction 61.

The user 23 may input patient-specific data 52 about the targeted skin lesion 51 into the three-dimensional mapping application 49. Ideally, this inputting is done via the computer interface 23 touch screen. However, other methods of inputting data could be used, such as an upload from the patient's electronic medical file. The patient-specific data 52 includes, but is not limited to, lesion size 54, lesion depth 55, and lesion location 56. The three-dimensional mapping application 49 uses the patient-specific data 52 input by the user 53 to create an enhanced three-dimensional reconstruction 57 of the targeted lesion 51.

The original three-dimensional reconstruction 50 captures information about the targeted skin lesion such as size, depth, and location. However, inputting the patient-specific data 52 to create an enhanced three-dimensional reconstruction 57 of the targeted lesion 51 increases the accuracy of the three-dimensional reconstruction 57. For purposes of this application, enhanced three-dimensional reconstruction means a three-dimensional reconstruction that has been augmented by additional data to increase accuracy. The step of creating an enhanced three-dimensional reconstruction 57 is optional. The steps described herein regarding three-dimensional reconstruction can be performed with either the original three-dimensional reconstruction 50 or the enhanced three-dimensional reconstruction 57.

The three-dimensional mapping and treatment system 20 is capable of generating and locally transmitting real-time data 60 comprising a comprehensive data set 58 for the targeted skin lesion. The comprehensive data set 58 includes, but is not limited to, a real time three-dimensional reconstruction 61 of the skin lesion. For purposes of this application, real-time data means information that is delivered immediately after collection. For purposes of this application, locally transmitting means the three-dimensional mapping and treatment system 20 is capable of generating, transmitting, and displaying data without sending the data to a remote network or cloud.

Preferably, the robotic arm 24 is a computer controlled robotic arm. A person of ordinary skill in the art understands that robotic arm medical devices are well known in the field. The computer that controls the robotic arm could be the computer 22 of the three-dimensional mapping and treatment system 20, or a separate computer integrated with the robotic arm 24. The robotic arm 24 is designed for precision and dynamic movement. The robotic arm 24 is designed to mimic the actions of a skilled surgeon. More specifically, the robot arm uses optimal treatment paths based on the intricacies of each targeted lesion.

The present invention comprises a first precision laser 25. The first precision laser 25 delivers laser therapy 59 to a target lesion 51. The nature of the laser therapy 59 is determined based on the real time three-dimensional reconstruction 60 of the targeted lesion. Alternatively, the laser therapy could be based on a non-real-time three-dimensional reconstruction of the targeted lesion 51.

The comprehensive data set 58 for the target lesion 51 collected by the three-dimensional mapping and treatment system 20 is used to determine an appropriate laser treatment 59 for the target skin lesion 51. Different frequencies 65 and colors 66 of lasers are utilized in order to precisely target specific aspects of the lesion such as depth 68, tissue type 69, and pathological features 70. The process of determining the appropriate laser treatment 59 can be made manually by the user or automated according to the steps described herein.

The three-dimensional mapping and treatment system 20 comprises a database of skin lesion treatment data 67. The database of skin lesion treatment data 67 comprises historic successful treatment data 98 and evidence-based treatment data 99 such as medical research, medical text books and other medical literature. The database of skin lesion treatment data 67 correlates laser characters such as color 66 and frequency 65 with lesion characteristics such as type, depth 68, tissue type 69, and lesion pathological features 70.

The three-dimensional mapping and treatment system 20 compares the comprehensive data set 58, or subsets of data from the comprehensive data set 58, with the database of skin lesion treatment characteristics 67. The three-dimensional mapping and treatment system 20 identifies treatment characteristics that correlate to the lesion characteristics contained with the data set 58. The three-dimensional mapping and treatment system 20 creates a unique treatment instruction 62 based on the treatment characteristics identified as correlating to the lesion characteristics in the data set 58.

The three-dimensional mapping and treatment system 20 transmits the unique instructions 62 to the robotic arm 24. The unique instructions 62 comprise laser frequency 100, laser power 101, laser color 102, and optimal treatment paths 103. The optical treatment paths 103 include specific positioning coordinates 104 for the robotic arm 24. The robotic arm 24 uses the instructions 62 from the three-dimensional mapping and treatment system 20 to guide delivery of laser treatments 59. The robotic arm 24 will move the laser according to the determined optimal treatment path 103 and positioning coordinates 104.

The three-dimensional mapping and treatment system may have a manual approval feature wherein the instructions 62 can be altered and approved by a user prior to delivering treatment.

The three-dimensional mapping and treatment system 20 is capable of transmitting real-time instructions 63 to the robotic arm 24. As the treatment 59 is provided, the characteristics of the targeted skin lesion 51 will change. The three-dimensional mapping and treatment system 20 is gathering and transmitting data from the targeted skin lesion 51 in real-time. The three-dimensional mapping and treatment system 20 can provide real-time instructions 63 that include updates and improvements form the original instructions 62. The real-time instructions 63 from the three-dimensional mapping and treatment system 20 enhances the accuracy of the robotic arm 24.

The laser treatment 59 may be automated wherein the robotic arm delivers the laser treatment 59 according to the instructions 62 63 provided by the three-dimensional mapping and treatment system 20. Optimal treatment paths 103 are calculated based on the three-dimensional reconstruction 61, 50 or 57. Alternatively, the user can manually perform the laser therapy 59 by controlling the robotic arm 24 using controls.

The laser treatment 59 is delivered in a manner similar to a three-dimensional printer. Each individual spot in the lesion 51 is treated based on the specific characteristics of that spot. The robotic arm 24 delivers meticulously planned treatment paths 103 guided by the robotic arm 24. Medical professionals can adjust treatment parameters on-the-fly, ensuring adaptability to the dynamic nature of lesions and patient responses. The present invention enhances repeatability and consistency across procedures, leading to improved treatment outcomes.

The present invention comprises a user-friendly computer interface 23. The user-friendly computer interface 23 could include, but is not limited, a touch screen 71 wherein the user 53 can input and read data, a voice activated controller 72 wherein the medical professional can orally dictate and hear data, and/or a conventional computer interface wherein the medical professional can type and read data. Preferably, all elements of the invention are controlled through a single user interface 23. In other embodiments, there may be multiple user interfaces for different components of the invention.

A person of ordinary skill in the art understands the present invention comprises suitable computer processing units, controllers, circuitry, wiring, and/or power supplies. A person of ordinary skill in the art understands the present invention comprises suitable computing capabilities to save, store, receive, process, and transmit data.

The present invention comprises the ability to integrate with existing electronic medical record systems 73 to ensure efficient data transfer and documentation. A person or ordinary skill in the art understands that suitable computer processing units, servers, software, wirelesses capabilities, circuitry, wiring and/or computing devices may be incorporated into the present invention to facility such integration.

The present invention allows for real-time adaptability and customization. The device allows for real-time adjustments based on the evolving condition of the lesion during treatment. Medical professionals can customize treatment parameters with ease, tailoring therapy to the unique characteristics of each patient's lesion.

The present invention provides many benefits over current lesion treatment practices and products. The integration of a computer-controlled robotic arm ensures unparalleled precision in treatment delivery, surpassing manual methods. Precise targeting of the lesion minimizes the risk of incomplete treatment or damage to healthy tissue, enhancing patient safety.

In some embodiments, the present invention further includes the use of artificial intelligence to assist with diagnosing skin lesions, determining appropriate treatments for skin lesions, and performing treatments on skin lesions.

Generally speaking, artificial intelligence relies on a dataset to predict outcomes. Here, an artificial intelligence dataset comprises data correlating historic skin lesion data 77 with (a) which aspects of the historic skin lesion data correlated to benign 78 or malignant 79 skin lesions; and (a) treatment data for successful treatment 80 of skin lesions having a specific skin lesion data pattern 81 (or a subset of unique skin lesion data indicator(s) 82).

In the present invention, data collected 83 by the three-dimensional mapping system 20 for a target skin lesion 51 is transmitted to an artificial intelligence platform 84. The artificial intelligence platform 84 compares the collected skin lesion data 83 with a historic skin lesion dataset 77 and predicts 85 whether the collected data set matches a benign or a malignant skin lesion. The artificial intelligence platform 84 also compares the collected skin lesion data 83 to a historic data set of successful skin lesion treatments 80 to find successful treatment data for skin lesion data that matches 86 (or is similar to) the collected skin lesion data 83.

The artificial intelligence platform 84 may communicate its results, including but not limited to suggested treatment data 87, to a computing device 22 in a screen readable format. In some embodiments, the artificial intelligence platform 84 may communicate its suggested treatment data 87 to the robotic arm 24 and/or the laser 25. The robotic arm 24 and/or the 25 laser are capable of receiving the treatment data 87, understanding the treatment data 87, and adjusting their settings to match the suggested treatment data 87.

The present invention may also consist of creating a skin lesion data set 88 for use as an artificial intelligence platform 84. More specifically, the skin lesion data 83 collected via three-dimensional mapping and the treatment data employed by the robotic arm and/or laser can be stored 84 to create a collected skin lesion data set 88 comprising collected malign lesion data 90 identifying characteristics of malign lesions and collected benign lesion data 91 identifying characteristics of benign skin lesions. The collected skin lesion data set 88 may further comprise collected successful treatment data 92 correlating treatments 93 that were used with the corresponding lesion characteristics 94. The present invention could use its own collected dataset 88 in the implementation of the artificial intelligence processes described above.

The present invention may further comprise a second hand-held laser device 27. In the embodiment shown in the figures, the second hand-held laser 27 is connected to the three-dimensional mapping and treatment system via cable 42. In other embodiments, the second laser 27 could be connected wirelessly. In other embodiments, the first laser of robotic arm may comprise a detachable portion (or be detachable in its entirety) to facilitate hand held lasering. The hand-held laser 27 allows a user to direct laser treatment 59 via hand if deemed necessary or advantageous when treating a patient.

The present invention's combination of three-dimensional mapping, robotic arm precision, laser therapy, and artificial intelligence is a paradigm shift in the treatment of skin lesions. Medical professionals can approach complex conditions such as skin cancer, chronic wounds, and scar tissue with unprecedented precision and customization. The present invention also helps avoid inadvertently damaging patient's skin. If an incorrect laser is used on a patient, burning and scarring could occur. By selecting a laser treatment based on real-time lesion characteristics, the risk of using incorrect laser characteristics is greatly reduced.

The present invention can also be implemented to provide cosmetic skin treatments. The present invention could be implemented in a line of different three-dimensional mapping and treatment systems for different purposes.

The integration of cutting-edge technologies results in improved outcomes for patients, with enhanced precision, safety, and customization to treatment delivery.

1. A skin treatment system comprising:
a three-dimensional mapping application, a robotic arm, a computer, a scanning probe, a database of laser treatments, and a precision laser.

2. The skin treatment system of claim 1 wherein:
the computer comprises a computer interface.

3. The skin treatment system of claim 2 wherein:
the robotic arm comprises a first end and a second end;
the first end of the robotic arm is connected to the computer;
the second end of the robotic arm extends outward from the computer; and
the scanning probe and the laser are located proximate the second end of the robotic arm.

4. The skin treatment system of claim 3 further comprising a second laser.

5. The skin treatment system of claim 4 wherein the second laser is removably connected to the robotic arm.

6. The skin treatment system of claim 3 wherein the robotic arm, the precision laser, the scanning probe, the computer, and the database are in electronic communication.

7. The skin treatment system of claim 6 further comprising a vacuum and a filter.

8. The skin treatment system of claim 7 further wherein the computer interface is a touch screen.

9. A method of treating a skin lesion with a laser comprising:
identifying a target skin lesion on a patient;
positioning a terminal end of a robotic arm near the target skin lesion, said terminal end of the robotic arm comprising a scanning probe and a precision laser;
creating a three-dimensional reconstruction of the target skin lesion using the scanning probe and a three-dimensional mapping application;
displaying the three-dimensional reconstruction of the skin lesion via a computer interface of a computer;
obtaining a data set for the target skin lesion comprising skin lesion characteristics for the target skin lesion;
comparing the data set for the skin lesion with a database of skin lesion treatment data accessible via the computer;
identifying skin lesion treatment data that corresponds to the skin lesion characteristics for the target skin lesion;
creating a laser treatment instruction for the target skin lesion based on the identified skin lesion treatment data corresponding to the skin lesion characteristics for the target skin lesion;
communicating the laser treatment instruction to the robotic arm;
the robotic arm performing laser treatment to the target skin lesion according to the laser treatment instruction.

10. The method of treating a skin lesion with a laser of claim 9 wherein the skin lesion characteristics for the target skin lesion comprise lesion size, lesion depth, lesion location, and tissue type.

11. The method of treating a skin lesion with a laser of claim 10 wherein the laser treatment instruction comprises laser color, laser power, laser frequency, and optimal treatment paths.

12. The method of treating a skin lesion with a laser of claim 11 wherein the optimal treatment paths comprise positioning coordinates for the robotic arm.

13. The method of treating a skin lesion with a laser of claim 9 wherein:
a real-time three-dimensional reconstruction of the targeted skin lesion is created and displayed via the computer interface a real-time data set for the target skin lesion is obtained;
a real-time comparison of the real-time data set for the target skin lesion is performed with the skin lesion treatment data;
a real-time identification of skin lesion treatment data corresponding to real-time data set for the target skin lesion is performed; and
and the laser treatment instructions for the target skin lesion are updated in real-time.

14. The method of treating a skin lesion with a laser of claim 9 further comprising the step of a review and approval of the laser treatment instruction prior to the step of communicating the laser treatment instruction to the robotic arm.

15. The method of treating a skin lesion with a laser of claim 9 further comprising a step of creating an enhanced three-dimensional reconstruction of the target skin lesion by inputting patient specific data into the three-dimensional mapping application.

16. The method treating a skin lesion of claim 9 wherein the database of skin lesion treatment data accessible via the computer comprises a database of historic skin lesion treatments and corresponding historic skin lesion characteristics.

17. The method of treating a skin lesion of claim 16 wherein an artificial intelligence platform compares the dataset of historic skin lesion treatments and corresponding historic skin lesion characters with the target skin lesion characteristics to create the laser treatment instruction for the target skin lesion.

18. The method of treating a skin lesion of claim 9 wherein the database of skin lesion treatment data comprises evidence based medical information on skin lesion treatment.

19. The method of treating a skin lesion of claim 9 further including the step of performing additional laser treatment using a second precision laser, wherein the second precision laser is removably attached to the robotic arm.

20. A skin treatment system comprising:
four legs;
a wheel at a bottom end of each leg;
a base atop a top end of the legs;
a computer housing atop the base;
a computer with a touchscreen computer interface inside the computer housing;
a robotic arm extending from the computer housing;
said robotic arm comprising a base section, a first arm section, a first elbow section, a second arm section, a second elbow section, a third arm section, a third elbow section, a fourth arm section, a fourth elbow section, and a termian end portion;
a first precision laser proximate the terminal end portion of the robotic arm;
a scanning probe proximate the terminal end portion of the robotic arm;
a vacuum proximate the terminal end portion of the robotic arm;
a second precision laser removably connected to the robotic arm;
a cable having a first end and a second end;
the first end of the cable connected to the second precisions laser;
the second end of the cable connected to the computer housing;
the computer, first precision laser, second precision laser, and scanning probe are in electrotonic communication;
a database of laser treatments accessible via the computer; and
a three-dimensional mapping application.
